(19) United States
(12) Patent Application Publication
(10) Pub. No.: US 2019/0321435 A1
(43) Pub. Date: Oct. 24, 2019

(54) ACORUS GRAMINEUS AS A PHARMACOLOGICALLY ACTIVE NATURAL TREATMENT IN THE ADDICTION OF NICOTINE

(71) Applicant: Rafael Valentin Hurtado, Providence, NC (US)

(72) Inventor: Rafael Valentin Hurtado, Providence, NC (US)

(21) Appl. No.: 16/390,980

(22) Filed: Apr. 22, 2019

Related U.S. Application Data
(60) Provisional application No. 62/660,466, filed on Apr. 20, 2018.

(51) Int. Cl. A61K 36/882; A61P 25/34; A61K 9/00; A61K 31/438; A61K 9/19

(52) U.S. Cl. CPC A61K 36/882; A61P 25/34; A61K 9/19; A61K 31/438; A61K 9/0053

(57) ABSTRACT

A plant-based, non-nicotine therapy for physiologically decreasing or eliminating the desire to use nicotine in humans by means of extracted Acorus gramineus is described. In particular, methods, pharmaceutical compositions, and kits for treating nicotine addiction by way of Acorus gramineus extracts are described. According to one example, Acorus gramineus rhizome water extracts are administered in an oral formulation containing 0.1 to 99% (w/w) water extract to a subject suffering from nicotine addiction.

### CROSS-REFERENCE TO RELATED APPLICATIONS

This application relies on the disclosure of and claims priority to and the benefit of the filing date of U.S. Provisional Application No. 62/660,466 filed Apr. 20, 2018, the disclosure of which is hereby incorporated by reference herein in its entirety.

### BACKGROUND OF THE INVENTION

### Field of the Invention

The present invention relates to compositions, methods, and kits for treating nicotine addiction. More particularly, embodiments of the present invention relate to formulations comprising a water extract or extracts of Acorus gramineus, kits including such formulations, and methods of treating nicotine addiction using such formulations.

### Description of Related Art

Plants of the family Acoraceae have been recognized for medicinal properties for thousands of years, and the active constituents and medical applications of plants of this family are still being characterized. Feng et al. describe phytochemical and neuropharmacological studies in relation to the numerous beneficial compounds extracted from the Acorus linnaeus family, as well as how rhizome extracts have been used in traditional medicine for a variety of purposes (see Feng, Xiao-Lin, Yang Yu, Da-Peng Qin, Hao Gao, and Xin-Sheng Yao. “Acorus Linnaeus: a review of traditional uses, phytochemistry and neuropharmacology.” RSC Advances 5, no. 7 (2015): 5173-182 (“Feng et al., 2015”)). The extracted Acorus rhizomes and their components, “particularly α- and β-asarone, possess anticonvulsant, antiepileptic, neuroprotective, memory enhancing and sedative properties.” Specifically, Acorus gramnieus has been documented for the treatment of cognitive decline (see Feng et al., 2015). A phytochemical investigation by Lee et al. resulted in the discovery of three previously unidentified quinone derivatives, shown in FIGS. 1A-C (see Lee, Sy, Jy Oh, Su Choi, and Kr Lee. “Quinone derivatives from the rhizomes of Acorus gramineus.” Biosci. Biotechnol. Biochem. 77, no. 2 (Feb. 23, 2013) (“Lee et al., 2013”)). Lee also described the anti-neuroinflammatory effects of the quinone derivatives, as well as their cytotoxicicity against four human tumor cell lines (see Lee et al., 2013).

Apomorphine is an aporphine derivative that activates D₁ through D₅ receptors, serotonin receptors and α-adrenergic receptors and therefore is a non-selective dopamine agonist (chemical structure shown in FIG. 2); apomorphine is structurally similar to dopamine (shown in FIGS. 3A and B). The use of apomorphine in human medicine for the treatment of addiction (such as heroine, alcohol and cigarettes), amongst other conditions, is mentioned by Ribarič (see Ribarič, Samo. “The Pharmacological Properties and Therapeutic Use of Apomorphine.” Molecules 17, no. 12 (2012): 5289-309 (“Ribarič, 2012”)).

According to the U.S. Centers for Disease Control and Prevention (CDC), tobacco use remains the single most preventable cause of mortality and morbidity in the United States. Tobacco's active component, nicotine, ranks as one of the most addictive substances known to man, and the advent and popularity of other alternative forms of nicotine administration such as electronic cigarettes, vaping, hookahs, etc. particularly among millennials is creating a new generation of addicted consumers. Available treatments for nicotine addiction include behavioral treatments as well as medications such as nicotine replacement therapy, bupropion and varenicline (CHANTIX®), the latter of which is believed to act by both stimulating nicotine receptors and preventing binding of nicotine to these receptors. However, these medications have side effects and contraindications which make them not appropriate for all patients. As with any disease, there is a need for new treatments with novel pharmacological mechanisms of action to provide both the medical practitioner and the patient with additional options that overcome these shortcomings.

### SUMMARY OF THE INVENTION

Embodiments of the present invention provide formulations of a water extract or extracts of Acorus gramineus that are useful for treating nicotine addiction, methods of treating nicotine addiction which include administration of such formulations to a patient suffering from nicotine addiction, as well as kits that can be used to treat nicotine addiction. Without committing to a particular theory, the water extracts and their active compounds are believed by the inventor to act by altering addictive and/or stereotypic behavior in humans with nicotine addiction, without acting on nicotine receptors.

Embodiments of the invention provide a pharmacognostic alternative treatment for nicotine addiction that is not based upon traditionally known pharmacological management. Additionally, therapeutic embodiments described herein are not expected to cause adverse effects, based on published phytochemical studies of the Acorus linnaeus genus. Furthermore, embodiments employing a route of administration by means of oral ingestion would be advantageous by effectively bypassing any issues that are oral-, dental- or dysphagia-related in consumers. These embodiments and their advantages will be further described in the foregoing Detailed Description.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIGS. 1A-C are chemical structures of three previously undiscovered quinone derivatives from the rhizomes of Acorus gramineus (from Lee, Sy, Jy Oh, Su Choi, and Kr Lee. “Quinone derivatives from the rhizomes of Acorus gramineus.” Biosci. Biotechnol. Biochem. 77, no. 2 (Feb. 23, 2013)).

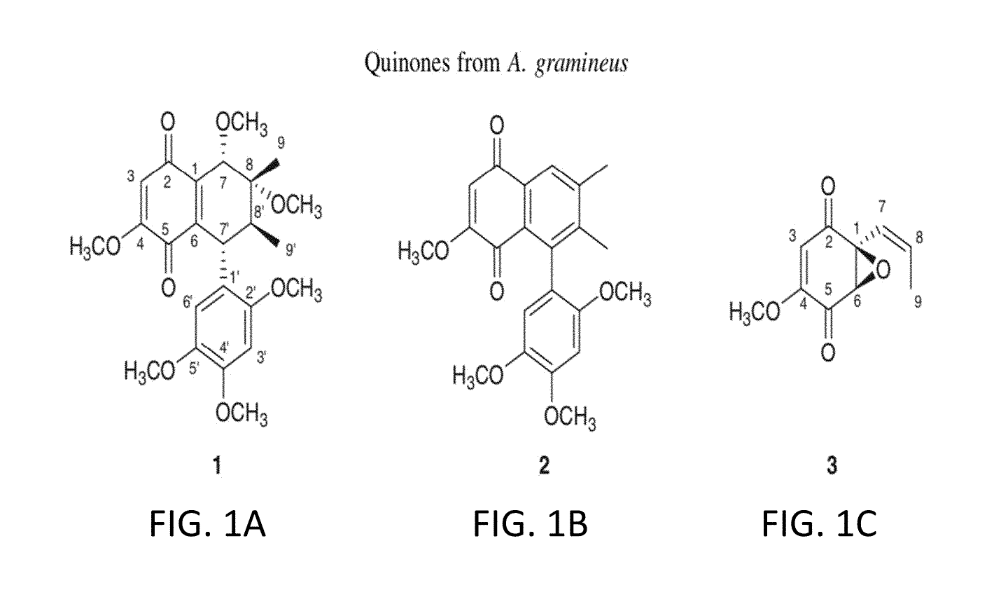

FIG. 2 is a chemical structure of apomorphine, a dopaminergic agonist that primarily affects the hypothalamic region of the brain.

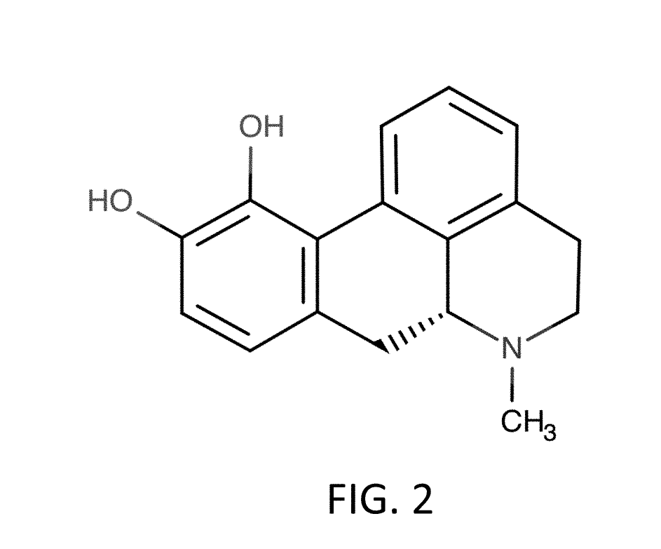

FIGS. 3A and B are chemical structures of dopamine hydrochloride and apomorphine hydrochloride, respectively (from Argiolas, A., and H. Hedlund. “The pharmacology and clinical pharmacokinetics of apomorphine SL.” BJU International 88 (2001): 18-21).

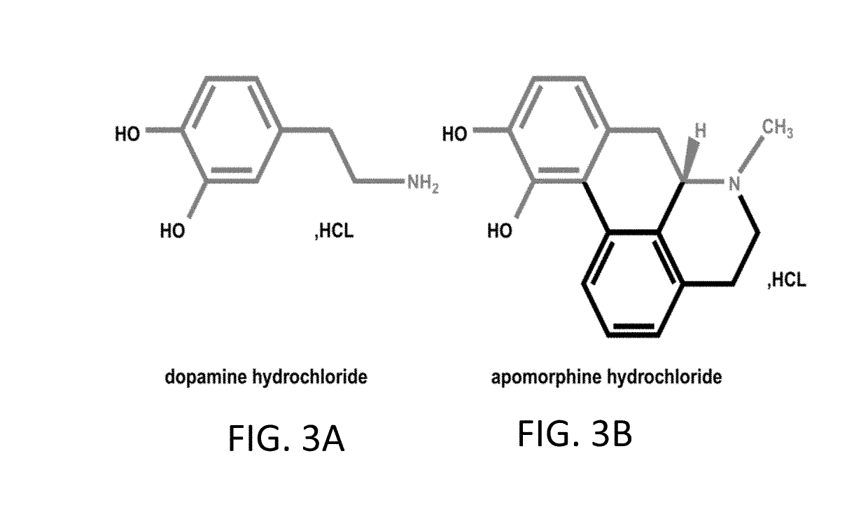

FIG. 4 is a chart showing biological properties displayed by water-extracted Acorus gramineus rhizomes.

FIGS. 5A and 5B are chemical structures of α- and β-asarone, respectively (from Feng, Xiao-Lin, Yang Yu, Da-Peng Qin, Hao Gao, and Xin-Sheng Yao. “Acorus linnaeus: a review of traditional uses, phytochemistry and neuropharmacology.” RSC Advances 5, no. 7 (2015): 5173-182).

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

As used herein, “nicotine addiction” is a disease mediated by stimulatory effects of nicotine on nicotinic cholinergic receptors in the brain and subsequent release of neurotransmitters such as dopamine, which is involved in drug-induced reward. The neuropharmacology and clinical effects of nicotine addiction has been reviewed (for example, see Benowitz N L. Nicotine Addiction. The New England journal of medicine. 2010; 362(24):2295-2303. doi:10.1056/NEJMra0809890, incorporated by reference). The term “nicotine addiction” and its grammatical variations includes any related condition, manifestation, sign or symptom such as physical dependence, reduced tolerance, changes in mood, withdrawal, craving, or any other physical, psychological, or behavioral manifestation of nicotine addiction. The term “nicotine addiction” includes such addiction as diagnosed by a medical practitioner, or self-diagnosed by a patient. However, the term “nicotine addiction” does not encompass other diseases or conditions that are epidemiologically associated with mortality or morbidity resulting from chronic inhalation of tobacco smoke or consumption of other tobacco products, such as cancer, cardiovascular disease, respiratory disease, or any other tobacco-related health risk recognized by the U.S. Centers for Disease Control and Prevention.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of nicotine addiction, as well as diminishment of extent of, delay, slowing, or prevention of progression, amelioration, palliation or stabilization, and other beneficial results described below. Symptoms of nicotine addiction can include any habitual behavior commonly associated with nicotine addiction, such as regular consumption of any tobacco product or any other product containing nicotine despite a strong desire to quit, as well as withdrawal symptoms upon cessation of nicotine intake such as cravings, increased appetite, headache, irritability, insomnia, restlessness, cold symptoms (e.g. cough, sore throat), gastrointestinal symptoms (e.g. nausea, constipation, diarrhea, stomach ache), and the like.

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, “reduction” of a symptom or symptoms (and grammatical equivalents of this phrase) means decreasing of the severity or frequency of the symptom(s), or elimination of the symptom(s).

As used herein, a “patient” is a human suffering from nicotine addiction or at risk for a nicotine addiction. A “subject” can also be such human, or can be an animal, such as an animal used in research or nicotine addiction or testing of therapies for treating nicotine addiction such as a mouse, rat, cat, dog, guinea pig, non-human primate, or sheep.

As used herein, an “effective amount” or a “therapeutically effective amount” of a drug or agent is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and can occur only after administration of a series of doses. Thus, a therapeutically effective amount can be administered in one or more administrations.

As used herein, a “therapeutically effective amount” of a drug can also be an amount of a drug that when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and can occur only after administration of a series of doses. Thus, a therapeutically effective amount can be administered in one or more administrations.

As used herein, “water extract” or “aqueous extract” are used interchangeably and includes any chemical compound, mixture of compounds, whether active or inactive, obtained from dissolving various constituents of Acorus gramineus using water as a solvent. The water extract can be an aqueous solution, or can be a concentrate, an oil, a solid, a crude power, a fine powder, a semi-liquid (gel or paste), a mixture, or any combination thereof, obtained from any water extraction procedure, including but not limited to the procedures described below. The term also encompasses extracts subject to one or more purification steps or other attempts to concentrate active ingredients, such as additional concentration, fractionation, distillation, or filtration steps.

As used herein, “rhizome” is what is commonly known in botany as a horizontal plant stem capable of producing the shoot and root systems of a new plant. While most rhizomes are situated underground, they may also be present at the soil surface or above ground.

As used herein, “active compound” or “active compounds” includes any compound demonstrated to be effective or potentially effective in treating nicotine addiction, such as in any clinical trial, test, experiment, assay, or procedure for assessing efficacy, whether performed in vivo, in vitro, or ex vivo. The active compound(s) can act individually, additively, or synergistically, by one or more mechanisms, such as an agonist, partial agonist, antagonist, of one or more receptors, whether or not previously characterized as having a role in nicotine addiction.

As used herein, in vitro indicates a procedure outside a living organism, ex vivo indicates a procedure involving removal of a cell, tissue, or organ from the body of a subject, and in vivo is within the body of a subject.

As used herein, “pharmaceutically acceptable carrier” means a material that is not biologically or otherwise undesirable, i.e., the material can be administered to a subject, along with the water extract or any compound thereof, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

As used herein, concentrations expressed as “% w/w” or “% (w/w)” indicates % weight per weight—which is known in the art as a concentration based on the weight of the individual component of the composition divided by the total weight of the composition. Unless explicitly stated otherwise, all concentrations disclosed herein are intended to be % weight per weight.

As used herein, “co-administration” of a therapy can mean the addition of a therapy that is prescribed by a physician, or self-administered by a patient or subject, to the patient's or subject's smoking cessation regimen. “Combination therapy” can mean the two therapeutic entities are co-administered, but in separate formulations, or formulated together (e.g., co-formulated in a single pill, capsule, etc.).

As used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Embodiments of the invention provide methods, compositions, and kits for treating nicotine addiction based on water extracts of Acorus gramineus.

According to one embodiment, the invention provides a method of treating a subject having an addiction to nicotine, the method including administering an effective amount of an Acorus gramineus water extract to the subject having the addiction to nicotine. The method can include one or more of the following features, in any combination, which are exemplified in further detail below in this specification, or any other feature provided in this specification:

The Acorus gramineus water extract can be obtained from an Acorus gramineus rhizome or alternatively from another part of the plant.

The Acorus gramineus water extract can be formulated in a pharmaceutically acceptable carrier such as an oral formulation.

The oral formulation can be a pill, capsule, lozenge, hard candy, or gum.

The oral formulation can include 0.1% to 99% w/w Acorus gramineus water extract.

The Acorus gramineus water extract can be administered at regular intervals or when the subject craves nicotine.

The method can also include administration of an effective amount of apomorphine.

According to another embodiment, the invention provides a composition including an Acorus gramineus water extract in a pharmaceutically acceptable carrier. The composition can include one or more of the following features, in any combination, which are exemplified in further detail below in this specification, or any other feature provided in this specification:

The Acorus gramineus water extract can be an Acorus gramineus rhizome water extract, or alternatively from other portions of Acorus gramineus.

The composition can be an oral formulation such as a pill, capsule, lozenge, hard candy, or gum.

The oral formulation can include 0.1% to 99% w/w Acorus gramineus water extract.

The oral formulation can optionally include apomorphine.

According to another embodiment, the invention provides a kit which includes a composition which includes an Acorus gramineus rhizome water extract in a pharmaceutically acceptable carrier and a set of written instructions for administering the composition. The kit can include one or more of the following features, in any combination, which are exemplified in further detail below in this specification, or any other feature provided in this specification:

The composition can be an oral formulation such as a pill, capsule, lozenge, hard candy, or gum.

The oral formulation or composition can include 0.1% to 99% w/w Acorus gramineus water extract.

According to embodiments of the methods, compositions, and kits of the invention, the Acorus gramineus water extract can be obtained by any procedure known in the art. Various procedures known in the art can be used to obtain a water extract of Acorus gramineus rhizomes, or any other part of Acorus gramineus. For example, the rhizomes can first be prepared for extraction by dissecting from the whole plant, and washing and drying them. The dried rhizomes can be further cut, minced, chopped, crushed and so on. In some embodiments, the dried pieces are further processed into ground or powdered samples to further increase surface contact with water. However, in other embodiments, fresh Acorus gramineus rhizomes or pieces thereof are used for the extraction. In other embodiments, the fresh Acorus rhizomes are put in a blender and minced prior to extraction.

To perform the water extraction, the prepared Acorus gramineus rhizomes can then be added to distilled water as solvent and subject to various extraction techniques. As an example only, approximately 100 g of crushed Acorus gramineus rhizomes is added to distilled water. Some of the extraction techniques available include maceration, infusion, digestion, decoction, percolation, hot continuous extraction (Soxhlet), aqueous alcoholic extraction by fermentation, counter-current extraction, and ultrasound extraction. These procedures can involve variations in temperature (e.g. heating the plant material up to the boiling point of the solvent, leaving it at room temperature, or heating at a temperature between room temperature and boiling), pressure, the prepared starting material (e.g. whether crushed plant material, course power, or fine powder is used) and the like. Autoclaving can subject the plant material to increased temperatures (i.e. greater than 100° C., such as 105-130° C.) as well as pressures greater than atmospheric pressure. Such procedures have been reviewed in the art (see Azwanida N N (2015) “A Review on the Extraction Methods Use in Medicinal Plants, Principle, Strength and Limitation”. Med Aromat Plants 4:196. doi:10.4172/2167-0412.1000196; and Sukhdev Swami Handa, Suman Preet Singh Khanuja, Gennaro Longo, Dev Dutt Rakesh (2008) “Extraction technologies for medicinal and aromatic plants”, International Centre for Science and High Technology (ICS)https://www.unido.org/sites/default/files/2009-10/Extraction_technologies_for_medicinal_and_aromatic_plants_0.pdf).

According to one embodiment, the water extraction procedure is as follows: dry Acorus gramineus rhizomes (e.g. 100 g) are boiled successively for 30 minutes in 600, 500 and 500 ml of twice-filtered water. The extracted solution is then filtered using filter paper and concentrated to 400 ml at 60° C., after which it is frozen and put through lyophilization. A suitable amount of powder extract is then dissolved in twice-filtered water to obtain a solution containing a particular amount of dry weight Acorus gramineus rhizomes per 1 ml solution. This is then centrifuged at 1000×g for 5 min. The resulting “water extract” is obtained from the supernatant (see Liao et al, Central inhibitory effects of water extract of Acori graminei rhizoma in mice. J Ethnopharmacol. 1998 July; 61(3):185-193 (“Liao et al., 1998”).

According to one embodiment, the water extraction procedure is according to the procedure as described in Liao et al. (e.g., pp. 185-193) (see Liao et al., 1998, incorporated by reference in its entirety).

According to some embodiments, other polar solvents can be added to, or used in substitution to, water for the extraction procedure, including other protic solvent such as methanol, ethanol, n-propanol, isopropanol, n-butanol, and the like, or aprotic solvents such as ethyl acetate, tetrahydrofuran (THF), acetone, dimethyl sulfoxide (DMSO) and the like. The other polar solvents can be added at a concentration of 5-95% of the solvent solution.

Any part of the Acorus gramineus plant can be used to obtain an extract, including any portion of the rhizome, stem, leaves, roots, shoots, stalks, fruits, flowers, seeds, and the like. However, in some embodiments, only Acorus gramineus rhizome water extracts are obtained.

According to some embodiments, the obtained water extract is subject to further processing to fractionate or isolate more potent fractions or compounds by conventional procedures well known in the art, (see Harborne J. B. Phytochemical methods: A guide to modern techniques of plant analysis, 3rd Ed. pp 6-7, 1998). For example, the water extract can be subject to fractionation using various columns known in the art, and the eluent used for fractionation can be chosen according to the column used. Non-limiting examples of adsorbents for fractionation columns include silica gel, alumina, and the like. The columns can also be functionalized to modify the surface polarity of the silica. Non-limiting examples of eluents include various organic solvents or organic solvent-water mixtures, the most common organic solvents including acetonitrile, acetone, methanol, hexane, pentane, cyclohexane, benzene, dichloromethane, chloroform, and ether.

According to some embodiments, the extracts and/or fractions can be subject to testing for toxicity and/or efficacy in various animal models. In one embodiment, the extracts and/or fractions are subject to testing for efficacy in a rodent model of nicotine addiction. Various rodent models of nicotine addiction have been established, including self-administration (SA), place conditioning (PC) and intracranial self-stimulation (ICSS), and have been reviewed (see Laura E. O'Della and Taline V. Khroyanb, “Rodent Models of Nicotine Reward: What do they tell us about tobacco abuse in humans?” Pharmacol Biochem Behay. 2009 February; 91(4): 481-488); Caille S et al., “Modeling nicotine addiction in rats” Methods Mol Biol. 2012; 829:243-56). For example, the extracts and/or fractions can be administered to laboratory rats (e.g. included in their feed, or administered by way of intraperitoneal or intravenous administration) during a test for intravenous self-administration (IVSA) of nicotine, and the effects of such extracts on self-administration behaviors can be gauged in comparison to a control group which is not administered the Acorus gramineus rhizomes water extracts. For example, high-dose bupropion was confirmed to reduce self-administration of nicotine in rodents (see Rauhut A S et al., “Effect of bupropion on nicotine self-administration in rats”, Psychopharmacology (Berl). 2003 August; 169(1):1-9. Epub 2003 Jun. 17).

The extract(s) and/or fractions can also be screened for toxicity using in vivo assays. For example, zebrafish allow for screening of fractions for toxicity in 96 well plates (see Challal S et al., “Zebrafish bioassay-guided microfractionation for the rapid in vivo identification of pharmacologically active natural products”. Chimia (Aarau) 66 (4), 229-232). The extract(s) and/or fractions can be subject to other batteries of tests (e.g. in vitro, ex vivo or in vivo) as known in the pharmaceutical arts, such as traditional rodent toxicity tests (e.g. LD50, histopathology); an exhaustive list need not be elaborated here.

The extracts can be formulated as part of a pharmaceutical composition comprising a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Compositions for oral administration include powders or granules, suspensions or solutions in water or non-aqueous media, as well as capsules, sachets, or tablets. Thickeners, flavorings, diluents, emulsifiers, dispersing aids or binders may be desirable. The pharmaceutical composition can also be in the form of ointments, elixirs, or injectable compositions. Pharmaceutically acceptable carriers include fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate. Auxiliaries are flow-regulating agents and lubricants, for example, silica, talc, stearic acid or salts thereof, such as magnesium stearate or calcium stearate, and/or polyethylene glycol. In one embodiment, dragee cores are provided with suitable coatings which, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules or nanoparticles which can optionally be mixed with fillers such as lactose, binders such as starches, and/or lubricants such as talc or magnesium stearate and, optionally, stabilizers. In one embodiment, the water extract is dissolved or suspended in suitable liquids, such as fatty oils, or liquid paraffin, optionally with stabilizers.

Fatty oils can comprise mono-, di- or triglycerides. Mono-, di- and triglycerides include those that are derived from C6, C8, C10, C12, C14, C16, C18, C20 and C22 acids. Exemplary diglycerides include, in particular, diolein, dipalmitolein, and mixed caprylin-caprin diglycerides. Triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof Exemplary triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

Pharmaceutical compositions comprising triglycerides can further comprise lipophilic and/or hydrophilic surfactants which can form clear solutions upon dissolution with an aqueous solvent. One such surfactant is tocopheryl polyethylene glycol 1000 succinate (vitamin E TPGS). Examples of such compositions are described in U.S. Pat. No. 6,267,985.

Possible pharmaceutical preparations which can be used rectally include, for example, suppositories, which consist of a combination of one or more of the extracts with a suppository base. Suitable suppository bases are, for example, natural or synthetic triglycerides, or paraffin hydrocarbons. In addition, it is also possible to use gelatin rectal capsules which consist of a combination of the water extract with a base. Possible base materials include, for example, liquid triglycerides, polyethylene glycols, or paraffin hydrocarbons. Suitable formulations for parenteral administration include aqueous solutions of the extract in water-soluble form, for example, water-soluble salts and alkaline solutions. In addition, suspensions of the extract as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400. Aqueous injection suspensions can contain substances which increase the viscosity of the suspension which include, for example, sodium carboxymethyl cellulose, sorbitol, and/or dextran. Optionally, the suspension can also contain stabilizers.

The topical compositions can be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). Emulsifiers, stabilizers, humectants and antioxidants can also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams may be formulated from a mixture of mineral oil, self-emulsifying beeswax and water in which extract, dissolved in a small amount of an oil such as almond oil, is admixed. A typical example of such a cream is one which includes about 40 parts water, about 20 parts beeswax, about 40 parts mineral oil and about 1 part almond oil.

Ointments can be formulated by mixing a suspension of the extract in a vegetable 25 oil such as almond oil with warm soft paraffin and allowing the mixture to cool. A typical example of such an ointment is one which includes about 30% almond oil and about 70% white soft paraffin by weight.

Lotions can be conveniently prepared by preparing a suspension of the extract in a suitable high molecular weight alcohol such as propylene glycol or polyethylene glycol.

Examples of antioxidants which can be added to the pharmaceutical compositions include BHA and BHT.

In certain embodiments, the water extracts of Acorus gramineus can be formulated in a chewing gum product. Chewing gums are typically composed of a gum base which provides its basic “chewable” matrix, as well as other ingredients such as flavors, colors, and sweeteners, and is formulated to gradually release such ingredients during sustained chewing. The chewing gum base can be any ingredient approved by the U.S. Food and Drug Administration as a chewing gum base (see U.S Code of Federal Regulations 21 CFR 172.615), such as natural ingredients including chicle, chiquibul, crown gum, gutta hang kang, massaranduba balata, massaranduba chocolate, nispero, rosidinha, venezuelan chicle, jelutong, Leche caspi (sorva), pendare, perillo, Leche de vaca, Niger gutta, Tunu (tuno), chilte, natural rubber, as well as synthetic ingredients such as butadiene-styrene rubber, isobutylene-isoprene copolymer (butyl rubber), paraffin, petroleum wax, petroleum wax synthetic, polyethylene, polyisobutylene, and polyvinyl acetate. The chewing gum product can also include softeners such as lecithin, hydrogenated vegetable oils, glycerol ester, lanolin, methyl ester, pentaerythritol ester, rice bran wax, stearic acid, sodium and potassium stearates. The chewing gum product can be formulated to provide sustained release of the extract of the invention while the product is chewed, such as 0.5 hrs, 1 hrs, 2 hrs, 3 hrs, 4 hrs, 5 hrs, or more.

Sweeteners for gum formulations can include such common ingredients as sugar, dextrose, glucose or corn syrup, erythritol, isomalt, xylitol, maltitol, mannitol, sorbitol, lactitol, aspartame, acesulfame-K, saccharine, sucralose, neohesperidine, dihydrichalcone.

Hard candies are typically composed of a sugar base resulting from various syrups that are heated to boiling and then cooled to become stiff and brittle solids. Lozenges can be prepared or compounded by molding or compressing ingredients such as sugars to form a hard lozenge, polyethylene glycol (PEG) to form a soft lozenge, or gelatin to form a chewable lozenge. The techniques for preparing these products are well known in the confectionary and pharmaceutical arts and need not be elaborated here.

Flavoring ingredients for chewing gum, hard candy, or lozenge formulation can be any flavoring which adds palatability to the product. Common flavor ingredients include oil soluble flavoring agents such as spearmint oil, cinnamon oil, oil of wintergreen (methylsalicylate), peppermint oils, clove oil, bay oil, anise oil, eucalyptus oil, thyme oil, cedar leaf oil, oil of nutmeg, oil of sage, oil of bitter almonds, peanut butter flavor, chocolate flavor, rum flavor, cassia oil, cinnamon mint flavor, and water soluble flavor ingredients such as various fruit extracts such as pineapple, mango, apple, banana, blackberry, blueberry, strawberry, peach, plum, grape, orange, tangerine, grapefruit, passion fruit, kiwi, pomegranate, raspberry, lemon, lime, and the like, artificial flavor ingredients and the like.

Color sources for chewing gum, hard candy, or lozenge formulations can include any approved natural or artificial color or color enhancer or combinations thereof. Examples of natural food colors include anthocyanins, canthaxanthin, chlorophyll, charcoal, cochineal, iron oxide, paprika, saffron, turmeric, and the like. Artificial colors include dyes and lakes, with dyes being more soluble in water and lakes being dispersible in fats and oils. Examples of artificial food colors include FD&C Blue Nos. 1 and 2, FD&C Green No. 3, FD&C Red No. 3, FD&C Red No. 40, FD&C Yellow No. 5, and FD&C Yellow No. 6.

Other embodiments of the Acorus gramineus water extract formulations can include other types of standard oral formulations known in the pharmaceutical arts, such as beverages, tinctures, solutions, syrups, elixirs, and the like.

According to embodiments, pharmaceutical compositions can contain from 0.10% to 99% by weight of the extract. The extracts can be formulated at various dosages, such as 1 g/kg to 990 g/kg of extract/kg oral dosage product (whether it be a pill, capsule, lozenge, hard candy, or gum) including 1, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 525, 550, 575, 600, 625, 650, 675, 700, 725, 750, 775, 800, 825, 850, 875, 900, 990 g/kg or more of extract/kg product, or any range encompassing or including these values (or corresponding weight percentages, such as 0.1%, 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52.5%, 55%, 57.5%, 60%, 62.5%, 65%, 67.5%, 70%, 72.5%, 75%, 77.5%, 80%, 82.5%, 85%, 87.5%, 90%, 99% or more of the oral dosage product, or any range encompassing of including these values).

The administered dose of Acorus gramineus extract to humans can be in the range 1 g/kg to 100 g/kg per day, including 1, 2, 3, 4, 5, 6, 7, 8, 9, 10, 15, 20, 25, 30, 35, 40, 45, 50, 55, 60, 65, 70, 75, 80, 85, 90, 95, or 100 g/kg per day.

The pharmaceutical compositions can be prepared at compounding pharmacies or manufactured industrially according to pharmaceutical methods known in the art. According to one embodiment of the invention, a kit is provided which includes 1) one or more of the oral dosage products described herein and 2) a set of written instructions for consuming the oral dosage product. For example, the written instructions can instruct the consumer to take the oral dosage product during periods of nicotine cravings or other withdrawal symptoms. Alternatively or in addition, the written instructions can instruct the consumer to take the oral dosage product at regular intervals such as once a day, twice a day, in the morning, before bedtime, with a meal, and so on. Such instructions can be provided according to optimal dosing schedules obtained from pharmacokinetic and pharmacodynamics studies of the Acorus gramineus water extract.

The extracts can be administered alone or in a pharmaceutical formulation such as those previously provided as examples. Compositions can be either in single or multiple dose forms. The amount of extract in any particular pharmaceutical composition will depend upon the effective dose, that is, the dose required to reduce a patient's consumption of nicotine. Suitable routes of administering the pharmaceutical compositions could include, but are not limited to, oral, rectal, topical (including dermal, buccal and sublingual), vaginal, or parenteral (including subcutaneous, intramuscular, intravenous, intradermal, intrathecal, epidural).

It will be understood by those skilled in the art that the route of administration will depend upon various pharmacokinetic factors such as the oral bioavailability of the active compounds, metabolism, half-life, distribution, etc. However, it is contemplated that oral administration of the water extracts and/or their particular active compound(s) in formulations such as a pill, capsule, candy, gum, or lozenge, would be most practical and have certain advantages with respect to efficacy.

The extract(s) or pharmaceutical compositions of the invention containing them can be administered to a subject in need thereof. The pharmaceutical compositions can be administered one or more times daily, either at prescribed intervals (e.g. before bedtime, in the morning, with or without meals), or as self-administered by the subject such as during periods of nicotine craving or during manifestations of nicotine withdrawal symptoms. In one embodiment, the extracts or pharmaceutical compositions containing them are administered orally. The extracts or pharmaceutical compositions can be administered at various dosages such as those provided above, according to the need of the subject. For example, dosages can be titrated upward until a therapeutic effect on nicotine addiction is achieved (e.g. cravings for nicotine are reduced).

According to some embodiments, the extract(s) or pharmaceutical compositions of the invention containing them can be co-administered, combined, and/or formulated with other therapies that are useful or suspected to be useful for treating nicotine addiction, as part of a smoking cessation regimen for a patient or subject, or as a product for smoking cessation.

Such therapies can include any of the following in any combination: nicotine itself (in the form of a nicotine replacement therapy, including but not limited to transdermal, chewing gum, lozenges, and inhalers); bupropion; other antidepressants, including but not limited to selective serotonin reuptake inhibitors (e.g. citalopram, escitalopram paroxetine, fluoxetine, fluvoxamine, sertraline), serotonin-norepinephrine reuptake inhibitors (e.g. desvenlafaxine, duloxetine, levomilnacipran, milnacipran, venlafaxine, desvenlafaxine), serotonin modulators (e.g. vilazodone, vortioxetine), serotonin antagonists and reuptake inhibitors (e.g. trazadone), norepinephrine reuptake inhibitors (e.g. vilazodone, vortioxetine), tricyclic antidepressants (e.g. amitriptyline, amitriptylinoxide, clomipramine, desipramine dibenzepin, dimetacrine, dosulepin, doxepin, imipramine, lofepramine, melitracen, nitroxazepine, nortriptyline, noxiptiline, opipramol, pipofezine protriptyline, trimipramine), tetracyclic antidepressants (e.g. amoxapine, maprotiline, mianserin, mirtazapine, setiptiline), mono-amine oxidase inhibitors (e.g. isocarboxazid, phenelzine, tranylcypromine, metralindole, moclobemide, pirlindole, toloxatone, bifemelane), natural or herbal antidepressants (e.g. St. John's Wort); atypical antipsychotics (e.g. aripiprazole, brexpiprazole, lurasidone, olanzapine, quetiapine, risperidone); varenicline; apomorphine; or any other therapy that is used or studied for smoking cessation at the time of or after the filing of this specification.

According to one particular embodiment, the extract(s) or pharmaceutical compositions of the invention containing them are co-administered, combined, or formulated with apomorphine as a smoking cessation regimen or product for smoking cessation. In such an embodiment, it may also be desirable to co-administer, combine or formulate an antiemetic with the apomorphine. Examples of antiemetics include domperidone, diphenhydramine, ondansetron, and diphenidol.

Co-formulation of active pharmaceutical ingredients is well known in the art. According to embodiments, the extract(s) are co-formulated in a pharmaceutical composition containing 2, 3, 4, or more of any of the agents mentioned above. Such co-formulations can include any of the oral formulations described herein, including a gum product, a hard candy, a lozenge, a pill, a capsule, a tablet, and the like.

According to some embodiments, the extracts or pharmaceutical compositions of the invention are co-administered with non-pharmacological therapies for smoking cessation, including but not limited to cognitive-behavioral therapy (CBT), counseling, and transcranial magnetic stimulation.

The following describes possible mechanisms of action for proposed therapeutic effects of water extract(s) of Acorus gramineus for treating nicotine addiction. However, such theory should not be construed as limiting or binding upon any interpretation of the claims.

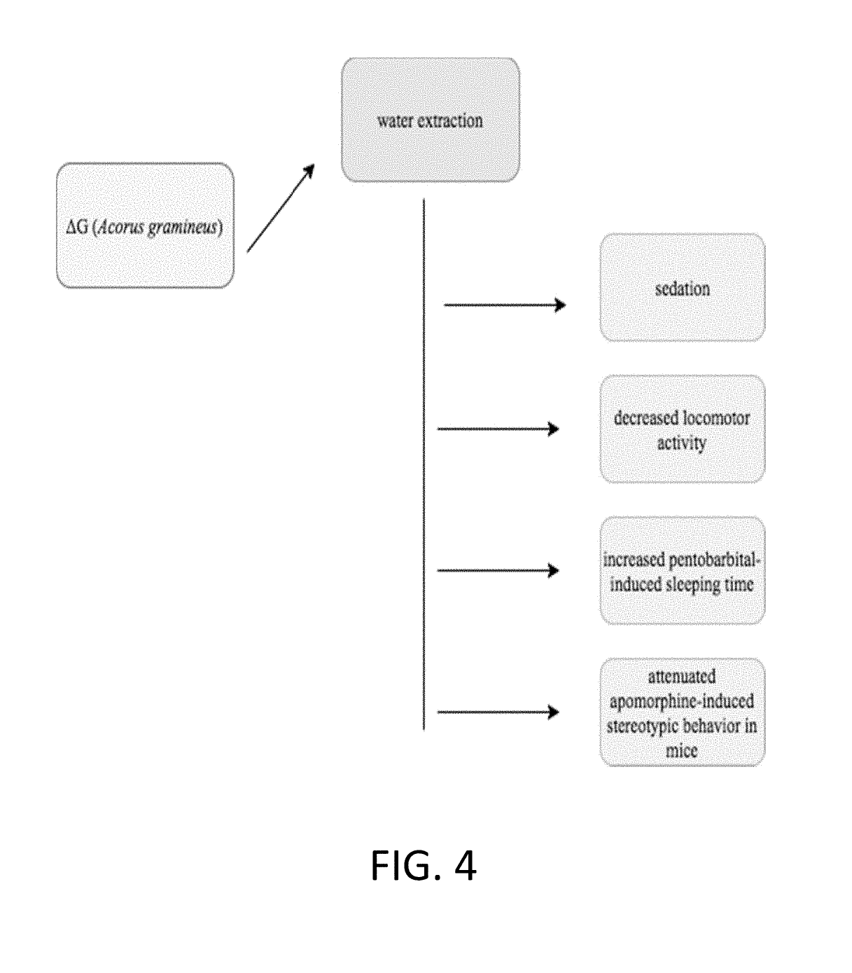

Water extraction of the Acorus gramineus rhizomes has been noted to exhibit various biological functions, including sedation, decreased locomotor activity and attenuated apomorphine-induced stereotypic behavior in mice (see Liao et al.). The central inhibitory effects of water extraction of Acorus gramineus rhizomes were evaluated by Liao et al. It was found that water extracts of Acorus gramineus rhizomes (0.5-5.0 g/kg) dose-dependently decreased the locomotor activity and also inhibited the intensity of apomorphine-induced stereotypic behavior, among other clinical effects, as shown in FIG. 4 (see Liao et al., 1998). Based on Liao et al.'s work, it appears that the central inhibitory effects of Acorus gramineus rhizomes water extracts were likely brought about by means of action on the central dopamine (D1 and D2) and GABA_{A} receptors. Dopamine D2 receptors are found in the in both the limbic and striatal regions of the brain, as well on sympathetic nerve terminals and cause vasodilatation by inhibiting noradrenaline release. It is important to note that the D2 receptor is the main receptor for all anti-psychotic drugs (see Crocker, Ann D. “Dopamine—mechanisms of action.” Experimental And Clinical Pharmacology 17, no. 1 (Jan. 1, 1994)).

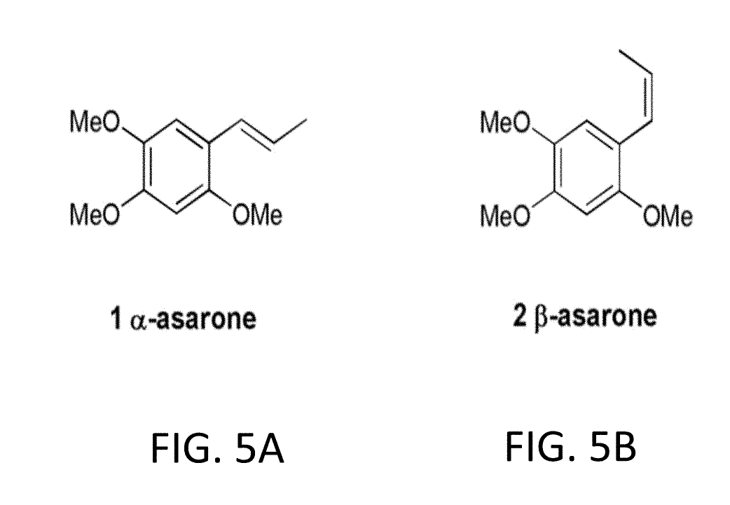

One objective for the invention is its production by means of oral formulation, whether it is a gum, hard candy, lozenge, tablet, or capsule. One importance of oral formulation (specifically gum or hard candy) of the product has to do with its action on apomorphine, which if administered intravenously can cause crystallization of the drug and lead to thrombus formation and pulmonary embolism (see Ribarič, 2012). Oral administration (specifically resulting from swallowing and ensuing metabolic breakdown in the stomach), on the other hand, is also not recommended due to apomorphine's significant first-pass hepatic metabolism and poor bioavailability (see Ribarič, 2012). In relation to the options of oral formulation, a gum or hard candy would provide more options for consumers to choose from, while also not compromising the absorption and efficacy of the intended product. This can be achieved by means of various natural flavorings added to any baseline formulation for gum/hard candy. The majority of the bioactivities of the Acorus species have been linked to the major active constituents, α- and β-asarone (structures shown in FIGS. 5A and 5B). In order to minimize the result of the opposing effects of α-asarone, methods such as carboxylation, hydroxylation and epoxidation have resulted in the discovery of 15 asarone analogues. These analogues have minimized “the contrasting effects of α-asarone, i.e., its efficient therapeutic potential and toxicity” (see Feng et al., 2015).

### EXAMPLES

The following hypothetical examples are intended to further illustrate the invention. However, they should not be construed as limiting any interpretation of the claims.

### Example 1

A first group of laboratory rats is administered an intraperitoneal injection of Acorus gramineus rhizomes water extract dissolved in saline (2 g extract/kg body weight). A second group of laboratory rats is administered pure saline intraperitoneally as a control. A third group of laboratory rats is administered bupropion dissolved in saline via intraperitoneal injection (70 mg/kg) as a positive control. All three groups are then tested for their frequency of intravenous self-administration (IVSA) of nicotine. It is found that the rodents administered the Acorus gramineus rhizomes water extract showed a marked reduction in IVSA of nicotine as well as the rodents administered bupropion, in comparison to the control group.

### Example 2A

Lozenges containing 10% w/w Acorus gramineus rhizomes water extract are prepared at a compounding pharmacy according to the instructions of a physician. The lozenges are then prescribed to a 47 year old male chronic smoker patient having a nicotine addiction of 8 years duration. The patient is primarily a consumer of tobacco cigarettes. The patient is instructed to consume the lozenges whenever he craves a cigarette. In a matter of weeks, the patient successfully quits smoking as a result of consuming the lozenges.

### Example 2B

The same patient as Example 2A, except the patient is prescribed capsules containing 25% w/w Acorus gramineus rhizomes water extract in a pharmaceutically acceptable carrier. The patient is instructed to take one capsule each day at bedtime. After a week of taking the capsules, the patient finds that he no longer craves a cigarette in the morning. Over a period of time, his consumption of cigarettes gradually decreases to a point where he feels he has successfully quit.

### Example 3

A 23 year old female has developed a nicotine addiction as a result of two years of regular vaping and desires to quit. She purchases an over-the-counter (OTC) gum product containing 20% w/w Acorus gramineus rhizomes water extract. The product includes written instructions to consume the gum during periods of nicotine cravings. The millennial finds that after chewing the gum for several hours, she no longer desires to vape.

The present invention has been described with reference to particular embodiments having various features. In light of the disclosure provided above, it will be apparent to those skilled in the art that various modifications and variations can be made in the practice of the present invention without departing from the scope or spirit of the invention. For example, a skilled artisan will recognize that the compositions, methods, and kits can be used as described or modified to be applied for treating other additive diseases and disorders, including opioid addiction, alcoholism, cocaine addiction, methamphetamine addiction, and the like. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure, including patents, published patent applications, and non-patent literature are each individually incorporated by reference herein in their entireties and as such are intended to provide an efficient way of supplementing the enabling disclosure of this invention as well as provide background detailing the level of ordinary skill in the art.

1. A method of treating a subject having an addiction to nicotine, the method comprising administering an effective amount of an Acorus gramineus water extract to the subject having the addiction to nicotine.

2. The method of claim 1, wherein the Acorus gramineus water extract is an Acorus gramineus rhizome water extract.

3. The method of claim 1, wherein the Acorus gramineus water extract is formulated in a pharmaceutically acceptable carrier.

4. The method of claim 1, wherein the Acorus gramineus water extract is formulated in an oral formulation.

5. The method of claim 4, wherein the oral formulation is a pill, capsule, lozenge, hard candy, or gum.

6. The method of claim 1, wherein the subject is a human.

7. The method of claim 4, wherein the oral formulation comprises 0.1% to 99% w/w Acorus gramineus water extract.

8. The method of claim 1, wherein the Acorus gramineus water extract is administered at regular intervals.

9. The method of claim 1, wherein the Acorus gramineus water extract is administered when the subject craves nicotine.

10. The method of claim 1, further comprising administration of an effective amount of apomorphine.

11. A composition comprising an Acorus gramineus water extract in a pharmaceutically acceptable carrier, wherein an amount Acorus gramineus water extract in the pharmaceutically acceptable carrier is capable of treating one or more signs, symptoms, or causes of nicotine addiction.

12. The composition of claim 11, wherein the Acorus gramineus water extract is an Acorus gramineus rhizome water extract.

13. The composition of claim 11, wherein the composition is an oral formulation.

14. The composition of claim 13, wherein the oral formulation is a pill, capsule, lozenge, hard candy, or gum.

15. The composition of claim 13, wherein the oral formulation comprises 0.1% to 99% w/w Acorus gramineus water extract.

16. The composition of claim 11, further comprising apomorphine.

17. A kit comprising:
a composition comprising an Acorus gramineus rhizome water extract in a pharmaceutically acceptable carrier, wherein an amount Acorus gramineus water extract in the pharmaceutically acceptable carrier is capable of treating one or more signs, symptoms, or causes of nicotine addiction; and
a set of written instructions for administering the composition.

18. The kit of claim 17, wherein the composition is an oral formulation.

19. The kit of claim 18, wherein the oral formulation is a pill, capsule, lozenge, hard candy, or gum.

20. The kit of claim 17, wherein the composition comprises 0.1% to 99% w/w Acorus gramineus water extract.

21. The method of claim 1, wherein the Acorus gramineus water extract is obtained by the following steps:
boiling Acorus gramineus rhizomes to obtain an extracted solution;
filtering, freezing, and lyophilizing the extracted solution to obtain a powder extract;
dissolving the powder extract in water to obtain a solution containing dissolved Acorus gramineus rhizomes; and
centrifuging the solution and obtaining a water extract from a supernatant which results from the centrifuging.
